(19) United States
(12) Patent Application Publication
(10) Pub. No.: US 2023/0076544 A1
(43) Pub. Date: Mar. 9, 2023

(54) SYSTEMS, METHODS, AND DEVICES FOR DAMAGING CANCEROUS CELLS BY APPLICATION OF ENERGY TO THE ENTIRETY OF THE CANCEROUS CELLS AND THE AREA OF THE BODY IMMEDIATELY SURROUNDING THE CANCEROUS CELLS

(71) Applicant: Howard Rosen, Montreal (CA)

(72) Inventor: Howard Rosen, Montreal (CA)

(21) Appl. No.: 18/053,719

(22) Filed: Nov. 8, 2022

Related U.S. Application Data
(63) Continuation-in-part of application No. 17/081,347, filed on Oct. 27, 2020.
Application No. 17/081,347 is a continuation of application No. 14/845,804, filed on Sep. 4, 2015, now abandoned.

(51) Int. Cl. A61B 18/12; A61N 7/02; A61N 1/40; A61B 18/06; A61F 7/00

(52) U.S. Cl. CPC A61B 18/12; A61N 7/02; A61N 1/403; A61B 18/06; A61F 7/007; A61B 2018/00041

(57) ABSTRACT

An apparatus for damaging or killing cancer cells in a living human body comprising a temperature monitoring apparatus that allows a user to monitor the temperature of a tumor and the surrounding tissue, such that when the tumor is at a temperature greater than the surrounding tissue, either because that is the nature of the tumor or because the tumor temperature has been raised through the introduction of an organic substance, an energy source may be used to elevate the temperature of the tumor above a critical temperature that damages or kills the tumor, but does not kill the surrounding non-cancerous cells because they start off at a lower temperature and are never allowed to go above the critical temperature that damages or kills those non-cancerous cells.

### CROSS-REFERENCE TO RELATED APPLICATIONS

This Application is a Continuation-In-Part of U.S. Non-Provisional patent application Ser. No. 17/081,347, filed on Oct. 27, 2020, titled “DAMAGING CANCEROUS CELLS UTILIZING RADIO FREQUENCY WAVES IN HEATING WITH HEATING ENHANCED BY INFUSION OR INJECTION OF GLUCOSE”, which is a Continuation of U.S. Non-Provisional application Ser. No. 14/845,804, filed on Sep. 4, 2015, titled “DAMAGING CANCEROUS CELLS UTILIZING RADIO FREQUENCY WAVES IN HEATING WITH HEATING ENHANCED BY INFUSION OR INJECTION OF GLUCOSE”, the contents of both of which are expressly incorporated herein by this reference as though set forth in their entirety and priority to which is claimed.

### FIELD

This disclosure is related to systems, methods, and devices for damaging or killing cancer cells in a living human body comprising a temperature monitoring apparatus that allows a user to monitor the temperature of a tumor and the surrounding tissue, such that when the tumor is at a temperature greater than the surrounding tissue, either because that is the nature of the tumor or because the tumor temperature has been raised through the introduction of an organic substance, such as glucose, an energy source may be used to elevate the temperature of the tumor above a critical temperature that damages or kills the tumor, but does not kill the surrounding non-cancerous cells because they start off at a lower temperature and are never allowed to go above the critical temperature that damages or kills those non-cancerous cells.

### BACKGROUND

It is well known in the art that cancerous tumors, in at least some cases, are exothermic and exist at a higher temperature than the non-cancerous tissue surrounding the tumor. For example, in the Sarcoma page of the Johns Hopkins Medicine website, https://www.hopkinsmedicine.org/health/conditions-and-diseases/sarcoma, it is indicated that soft tissue sarcomas may include swelling that may “feel warm to the touch.” This publicly available webpage is incorporated by reference into this disclosure as though set forth in its entirety. Additional in the National Library of Medicine of the National Institute of Health article titled “Screening through Temperature and Thermal Pattern Analysis in DMBA-Induced Breast Cancer in Wistar Rats”, found at https://www.ncbi.nlm.nih.gov/pmc/articles/PMC8385218/, indicates that normal breast tissue is at 36.66 C, whereas cancerous breasts are at 38.87 C. The results revealed significantly higher heat in breasts with cancerous lesions. This publicly available webpage is incorporated by reference into this disclosure as though set forth in its entirety. Further, the ResearchGate reference found at https://www.researchgate.net/figure/Dynamic-thermal-analysis-in-a-patient-with-T1-breast-cancer_fig27916759, states:
that Temperature in a normal breast increases from the skin into the deep tissue and heat conductivity in the healthy breasts is constant in most cases and generally can be characterized in terms of circadian rhythm periodicity [29]. In contrast, the rhythms associated with malignant cells proliferation are largely non circadian and suggest that a circadian to ultradian shift may be a general correlation to neoplasia. Heat production by the tumour under the influence of angiogenesis should be therefore re-examined in terms of absence of normal circadian fluctuations. Due to the increased blood flow and the lack of receptivity in the newly formed vessels in malignancy, temperature production exhibits circadian rhythmic variations to a far lesser degree than is evident in the healthy breasts [13]. It has been found that independent of a tumour's size, relatively small tumours (>/=0.5 cm in diameter), poorly vascularized rapidly growing tumors can produce increases in regional heat. The explanation for this effect is unclear but it may be due to the chronic inflammatory response around developing breast tumours.
   These references are just three of many that support the well known fact that some tumors have an elevated temperature with respect to surrounding tissue.

### SUMMARY

To minimize the limitations in the prior art, and to minimize other limitations that will become apparent upon reading and understanding the present specification, the present disclosure is directed to devices and methods for damaging cancerous cells that employs a temperature monitoring apparatus which controls an energy emitting apparatus to direct energy on the entirety of a tumor, and to the area immediately around the tumor, without killing the surrounding tissue. This is because a tumor may be at a temperature greater than the surrounding tissue, either because that is the nature of the tumor or because the tumor temperature has been raised through the introduction of an organic substance, such that an energy source may be used to elevate the temperature of the tumor above a critical temperature that damages or kills the tumor, but does not kill the surrounding non-cancerous cells because they start off at a lower temperature and are never allowed to above the critical temperature that damages or kills those non-cancerous cells.

The present disclosure relates to treatment of cancer by the killing or damaging of cancer cells utilizing various energy-based therapies, such as hyperthermia or radiation therapy. The energy-based therapy for the systems, methods, and devices of the present disclosure may be employed by an energy emitter. A temperature monitoring device may control, or may provide feedback that allows a controller to control, the energy emitter to direct energy to the tumor, and the area immediately around the tumor, while not killing the surrounding tissue. In this disclosure tumor may be used interchangeable with cancerous cells, mass, or growth. The temperature monitoring apparatus may take a plurality of temperature measurements and monitor for changes to those measurements.

Tumors and/or cancerous cells tend to exist at a higher temperature than surrounding tissue because abnormal cell architecture and angiogenesis. More specifically, tumors have poor heat dissipation and tend to be hotter than surrounding tissue due to inflammation, higher metabolic rate, interstitial hypertension, abnormal vessel morphology, and lack or response to homeostatic signals, among other factors. The surrounding (non-cancerous) tissue, lacking these factors, is consequently a more normal body temperature and at a temperature that is lower than the cancerous cells.

To minimize damage to healthy tissue and other adverse effects, the temperature of the tumor and/or the surrounding tissue may be monitored. The goal of the devices, systems, and methods of the present disclosure is to keep local temperatures in tumor bearing tissue under 44° C. (111° F.) to avoid damage to surrounding tissues. These temperatures have been derived from cell culture and animal studies. The body keeps itself normal human body temperature, near 37.6° C. (99.7° F.). Unless a needle probe can be placed with accuracy in every tumor site amenable to measurement, there is an inherent technical difficulty in how to actually reach whatever a treating center defines as an “adequate” thermal dose. Since there is also no consensus as to what parts of the body need to be monitored (common clinically measured sites are ear drums, oral, skin, rectal, bladder, esophagus, blood probes, or even tissue needles). Clinicians have advocated various combinations for these measurements. These issues complicate the ability of comparing different studies and coming up with a definition of exactly what a thermal dose actually should be for a particular tumor, and what dose may end up being toxic to different types of tissues in humans that are surrounding the tumors. The temperature monitoring may be accomplished by probes, sensors, or advanced imaging techniques. The temperature monitoring of the tumor and surrounding tissue may preferably be done in real time during a heat treatment via the advanced imaging techniques.

Various methods may be used to measure the temperature of a tumor and its surrounding tissue. There are both invasive and non-invasive methods of temperature monitoring. Some examples of invasive monitoring include thermocouples and fiber-optic sensors.

Advanced imaging techniques may be used as non-invasive temperature monitoring, including, but not limited to, magnetic resonance thermometry, computed tomography, infrared thermography, and photoacoustic imaging. Magnetic resonance thermometry relies on the technology that is used in magnetic resonance imaging (MRI), where an image is generated based on a temperature-dependent imaging signal. Computed tomography uses computerized x-ray imaging, which rotates a narrow beam of x-rays at a patient, generating cross-sectional images of the body. Photoacoustic imaging utilizes endogenous contrast between various tissues in vivo.

Infrared thermography is where non-invasive infrared thermal emissions are emitted from a device to determine the temperature and location of a tumor, using the temperature profile on the surface of the skin. A variation of this method may involve using the thermal-emission spectrum in the semitransparent spectral region of the target object to extract the tumor's temperature as a function of depth. Volumetric temperature distributions, which give a visualization of the variations in temperature for a given space, can be produced when combining this infrared thermography with two-dimensional imaging devices, such as an infrared hyperspectral camera or scanning a single-pixel spectrometer.

Another way to measure the temperature of a tumor and the surrounding non-cancerous tissue involves simultaneously measuring infrared radio brightness at different points of the body, using a multi-channel multi-frequency radiothermograph. This allows a more accurate localization of the heat source due to three-dimensional (3D) visualization of the internal heat field of a body, and monitoring the temperature dynamics of a heat point source inside the human body.

In another embodiment of the systems, methods, and devices of the present disclosure, these approaches can be used in conjunction with energy emitters to target and kill cancerous cells more successfully.

Researchers are also studying substances that help radiation better destroy tumors (radiosensitizers) and those that better protect healthy tissues near the area being treated (radioprotectors). These approaches can also be used in conjunction with injection or introduction of glucose (or other organic materials) and radiation to more precisely target and kill cancerous cells while leaving other cells unharmed or less harmed. Because cancer cells have a different energy metabolism, giving more sugar to cancer cells does not speed their growth, and likewise, depriving cancer cells of sugar does not slow their growth. But glucose is quickly and readily absorbed by cancer cells and tissues that are using more energy—including cancer cells—absorb greater amounts of glucose. As a consequence of the excess glucose absorption, the cancer cells create a heat byproduct, effectively raising the temperature of the cancer cells.

The 1931 Nobel laureate in medicine, German Otto Warburg, Ph.D., first discovered that cancer cells have a fundamentally different energy metabolism compared to healthy cells. The crux of his Nobel thesis was that malignant tumors frequently exhibit an increase in anaerobic glycolysis—a process whereby glucose is used as a fuel by cancer cells with lactic acid as an anaerobic byproduct—compared to normal tissues. Warburg hypothesized that cancer growth is caused by tumor cells mainly generating energy (as e.g. adenosine triphosphate/ATP) by anaerobic breakdown of glucose (known as fermentation, or anaerobic respiration). This is in contrast to healthy cells, which mainly generate energy from oxidative breakdown of pyruvate. Pyruvate is an end product of glycolysis, and is oxidized within the mitochondria. Utilizing more energy implies that cancer cells supplied with more glucose will absorb and burn that glucose at a higher rate than normal cells and therefore will heat up in comparison to normal cells or normal surrounding cells. That is, given injections of glucose, cancer cells will heat themselves to a temperature higher than normal cells or surrounding normal cells also exposed to the glucose.

Supplying further energy to heat both cancer cells and normal cells results in both the normal cells and the cancer cells to be heated above normal body temperatures with the cancer cells heating further above normal temperatures than the surrounding cells due to an infusion of glucose. This results in cancer cells being heated to a higher temperature than surrounding cells which has an increased negative effect on survival of cancer cells or damage to cancer cells in comparison to the survival of or damage to normal or surrounding cells. Thus, cancer cells are essentially targeted for damage/extinction by providing them with glucose (or other organic material) which causes them to heat up, and then also exposing them to further heating in another manner, such as a form of general radiation, or a targeted form of radiation directed primarily at the cancer cells or a specific region or area containing cancer cells so as to kill or damage the cancerous cells without killing or damaging nearby or surrounding cells (or causing less damage to “normal” cells than to cancerous cells).

Localized and whole-body application of heat has been proposed as a technique for the treatment of malignant tumors. Intense heating will cause denaturation and coagulation of cellular proteins, rapidly killing cells within a tumor. More prolonged moderate heating to temperatures just a few degrees above normal (39.5° C.) can cause more subtle changes. A mild heat treatment combined with other stresses can cause cell death by apoptosis. There are many biochemical consequences to the heat shock response within the cell, including slowed cell division and increased sensitivity to ionizing radiation therapy. The purpose of overheating the tumor cells is to create a lack of oxygen so that the heated cells become over-acidified, which leads to a lack of nutrients in the tumor. This in turn disrupts the metabolism of the cells so that cell death (apoptosis) can set in. In certain cases, chemotherapy or radiation that has previously not had any effect can be made effective. Hyperthermia alters the cell walls by means of so-called heat shock proteins. The cancer cells then react very much more effectively to the cytostatic treatments and radiation. If hyperthermia is used conscientiously, it has no serious side effects.

There are other techniques by which heat may be delivered. Some of the most common involve the use of focused ultrasound (FUS or HIFU), microwave heating, induction heating, magnetic hyperthermia, and direct application of heat through the use of heated saline pumped through catheters. One method involves carbon nanotubes that selectively bind to cancer cells. Lasers may then be used that pass light harmlessly through the body, but heat the nanotubes, causing the death of the cancer cells. Similar results have also been achieved with other types of nanoparticles, including gold-coated nanoshells and nanorods that exhibit certain degrees of ‘tunability’ of the absorption properties of the nanoparticles to the wavelength of light for radiation. The success of this approach to cancer treatment rests on the existence of an ‘optical window’ in which biological tissue (i.e., healthy cells) are completely transparent at the wavelength of the laser light, while nanoparticles are highly absorbing at the same wavelength. Such a ‘window’ exists in the so-called near-infrared region of the electromagnetic spectrum. In this way, the laser light can pass through the system without harming healthy tissue, and only diseased cells, where the nanoparticles reside, get hot and are killed.

Magnetic hyperthermia makes use of magnetic nanoparticles, which can be injected into tumors and then generate heat when subjected to an alternating magnetic field. One of the challenges in thermal therapy is delivering the appropriate amount of heat to the correct part of the patient's body. A great deal of current research focuses on precisely positioning heat delivery devices (catheters, microwave, and ultrasound applicators, etc.) using ultrasound or magnetic resonance imaging, as well as of developing new types of nanoparticles that make them particularly efficient absorbers while offering little or no concerns about toxicity to the circulation system. Clinicians also hope to use advanced imaging techniques to monitor heat treatments in real time—heat-induced changes in tissue are sometimes perceptible using these imaging instruments.

One non-invasive cancer treatment involves using radio waves to heat up tiny metals that are implanted in cancerous tissue. Gold nanoparticles or carbon nanotubes are the most likely candidate. Promising preclinical trials have been conducted.

Another method that is entirely non-invasive referred to as Tumor Treating Fields. This method applies an electric field through a tumor region using electrodes external to the body. Successful trials have shown the process effectiveness to be greater than chemotherapy and there are no side-effects and only negligible time is spent away from normal daily activities. This treatment is still in very early development stages for many types of cancer.

High-intensity focused ultrasound (HIFU) is still in investigatory phases in many places around the world. In China, it has received CFDA approval and over 180 treatment centers have been established in China, Hong Kong, and Korea. HIFU has been successfully used to treat cancer to destroy tumors of the bone, brain, breast, liver, pancreas, rectum, kidney, testes, and prostate. Several thousand patients have been treated with various types of tumors. HIFU has received CE approval for palliative care for bone metastasis. Experimentally, palliative care has been provided for cases of advanced pancreatic cancer.

Hyperthermia therapy in general is a type of medical treatment in which body tissue is exposed to slightly higher temperatures to damage and kill cancer cells or to make cancer cells more sensitive to the effects of radiation and certain anti-cancer drugs. Techniques that may bring local tissues to quite high temperatures, such as radio frequency ablation, are not usually meant by “hyperthermia.” When combined with radiation therapy, it is called thermos-radiotherapy.

Local hyperthermia has shown to be effective when combined with chemotherapy or radiation therapy for cancers such as breast, cervical, prostate, head and neck, melanoma, soft-tissue sarcoma and rectal cancer, among others. Whole-body hyperthermia is generally considered to be a promising experimental cancer treatment, but requires close medical monitoring of the patient, as side effects can be serious.

Hyperthermia is considered the “fourth leg” of cancer treatment. It was historically reserved for the most severe or recurrent cases of cancer. However, there is more evidence to support its use as a primary treatment, as is the practice in parts of Europe, including the Netherlands, Germany, and Austria. Regional hyperthermia is currently becoming more utilized in the United States and elsewhere, as devices come to market. Whole body hyperthermia, using radiant heat chambers is almost always used as an adjuvant therapy. The most effective uses are currently being studied.

Hyperthermia is defined as supra-normal body temperatures. There is no consensus as to what is the safest or most effective target temperature for the whole body. During treatment the body temperature reaches a level between 39.5 and 40.5° C. (103.1 and 104.9° F.). However, other researchers define hyperthermia between 41.8-42° C. (107.2-107.6° F.) (Europe, USA) to near 43-44° C. (109-111° F.). Temperature and time are interrelated, with longer times at temperature meaning more cancerous cells are killed but also higher risk of toxicity to the body. Hyperthermia may kill or weaken tumor cells, and is controlled to limit effects on healthy cells. Tumor cells, with a disorganized and compact vascular structure, have difficulty dissipating heat. Hyperthermia may therefore cause cancerous cells to undergo apoptosis in direct response to applied heat, while healthy tissues can more easily maintain a normal temperature.

Even if the cancerous cells do not die outright, they may become more susceptible to ionizing radiation therapy or to certain chemotherapy drugs. The heat with applied, local hyperthermia will dilate blood vessels to the tumor, increasing oxygenation of the tumor, thereby making radiation therapy more effective. Oxygen is a potent radio-sensitizer, increasing the effectiveness of a given dose of radiation by forming DNA-damaging free radicals. Tumor cells in a hypoxic environment may be as much as 2 to 3 times more resistant to radiation damage than those in a normal oxygen environment.

Energy emitters may also be employed in radiation therapy, where high-energy x-rays or other particles are used to destroy cancer cells.

One such energy emitting cancer therapy are radionuclide therapies. There are three basic radionuclide electron particle options, being alpha, beta, and Auger electron emission. Alpha particle electrons have a moderate pathlength and high linear energy transfer, allowing them to effectively treat small neoplasms and micrometastases. Beta particle electrons long particle pathlength and low linear energy transfer, making them effective at treating medium to large size tumors, but can result in the irradiation of healthy surrounding tissue. Lastly, Auger particle electrons have a high linear energy transfer and a limited pathlength, restricting their efficacy to single cells.

Another energy emitting cancer therapy is proton therapy, where a beam of protons deliver radiation directly to the tumor. The proton beam, when charged at a high energy, can effectively damage cancerous tissues while sparing healthy tissues and organs.

These forms of energy emitting therapies can be employed in several ways. One such way is with 3-dimensional conformal radiation therapy. With this method, detailed 3-dimensional pictures are created, allowing those treating the patient to aim the radiation therapy more precisely, allowing higher doses of radiation therapy while minimizing damage to surrounding tissues.

Another way to employ energy emitting therapies is with intensity modulated radiation therapy. With this method, the intensity of the radiation is varied within each field, more effectively targeting the cancerous tissue and minimizing harm to normal tissue.

Image guided radiation therapy is also employed by using daily images of each treatment field to confirm patient positioning and to make sure the target is in the field. These images are used to decrease the treatment field over time to better target the tumor and minimize damage to surrounding tissue.

Hyperthermia has also been proven to be effective when combined with chemotherapy. Published studies have shown an improvement of 10 year disease free survival in bladder cancer patients treated with combined hyperthermia and chemotherapy with 53% survival, versus those treated with chemotherapy alone, with 15% survival after 10 years.

Intense heating will cause denaturation and coagulation of cellular proteins, rapidly killing cells within a tumor. More prolonged moderate heating to temperatures just a few degrees above normal can cause more subtle changes. A mild heat treatment combined with other stresses can cause cell death by apoptosis. There are many biochemical consequences to the heat shock response within the cell, including slowed cell division and increased sensitivity to ionizing radiation therapy.

Hyperthermia can kill cells directly, but it is more important use is in combination with other treatments for cancer. Hyperthermia increases blood flow to the warmed area, perhaps doubling perfusion in tumors, while increasing perfusion in normal tissue by ten times or even more. This enhances the delivery of medications. Hyperthermia also increases oxygen delivery to the area, which may make radiation more likely to damage and kill cells, as well as preventing cells from repairing the damage induced during the radiation session.

Cancerous cells are not inherently more susceptible to the effects of heat. When compared in in vitro studies, normal cells and cancer cells show the same responses to heat. However, the vascular disorganization of a solid tumor results in an unfavorable microenvironment inside tumors. Consequently, the tumor cells are already stressed by low oxygen, higher than normal acid concentrations, and insufficient nutrients, and are thus significantly less able to tolerate the added stress of heat than a healthy cell in normal tissue.

Mild hyperthermia, which provides temperatures equal to that of a naturally high fever, may stimulate natural immunological attacks against the tumor. However, it is also induces a natural physiological response called thermo-tolerance, which may tend to protect the treated tumor. Moderate hyperthermia, which heats cells in the range of 40 to 42° C. (104to 108° F.), damages cells directly, in addition to making the cells radiosensitive and increasing the pore size to improve delivery of large-molecule chemotherapeutic and immunotherapeutic agents (molecular weight greater than 1,000 Daltons), such as monoclonal antibodies and liposome-encapsulated drugs. Cellular uptake of certain small molecule drugs is also increased. Most local and regional cancer treatments are in this temperature range. Very high temperatures, above 50° C. (122° F.), are used for ablation (direct destruction) of some tumors. This generally involves inserting a metal tube directly into the tumor, and heating the tip until the tissue next to the tube has been killed.

There are many techniques by which heat may be delivered. Some of the most common involve the use of focused ultrasound (FUS or HIFU), infrared sauna, microwave heating, induction heating, magnetic hyperthermia, infusion of warmed liquids, or direct application of heat such as through sitting in a hot room or wrapping a patient in hot blankets.

Local hyperthermia heats a very small area, usually the tumor itself. In some instances, the goal is to kill the tumor by heating it, without damaging anything else. The heat may be created with microwave, radiofrequency, ultrasound energy or using magnetic hyperthermia. Depending on the location of the tumor, the heat may be applied to the surface of the body, inside normal body cavities, or deep in tissue through the use of needles or probes. One relatively common type is radiofrequency ablation of small tumors. This is easiest to achieve when the tumor is on a superficial part of the body, which is called superficial hyperthermia, or when needles or probes are inserted directly into the tumor, which is called interstitial hyperthermia.

Regional hyperthermia heats a larger part of the body, such as an entire organ or limb. Usually, the goal is to weaken cancer cells so that they are more likely to be killed by radiation and chemotherapeutic medications. This may use the same techniques as local hyperthermia treatment, or it may rely on blood perfusion. In blood perfusion, the patient's blood is removed from the body, heated up, and returned to blood vessels that lead directly through the desired body part. Normally, chemotherapy drugs are infused at the same time. One specialized type of this approach is continuous hyperthermic peritoneal perfusion (CHPP), which is used to treat difficult cancers within the peritoneal cavity (the abdomen), including primary peritoneal mesothelioma and stomach cancer. Hot chemotherapy drugs are pumped directly into the peritoneal cavity to kill the cancer cells.

Whole-body hyperthermia heats the entire body to temperatures of about 39 to 43° C. (102 to 109° F.), with some advocating even higher temperatures. It is typically used to treat metastatic cancer, that is, cancer that has spread to many parts of the body. Techniques include infrared hyperthermia domes which include the whole body or the body apart from the head, putting the patient in a very hot room/chamber, or wrapping the patient in hot, wet blankets or a water tubing suit. Others have submerged patients in wax. Methods of pumping the blood outside of the body through heating elements have also been applied.

Moderate hyperthermia treatments usually maintain the temperature for about an hour or so.

The schedule for treatments has varied between study centers, as nobody knows what is most effective and treatment schedules have been made based on cell culture or animal studies, or simply been built around a course of planned chemotherapy. After being heated, cells develop resistance to heat, which persists for about three days and reduces the likelihood that they will die from direct cytotoxic effects of the heat. Some even suggest maximum treatment schedule of twice a week. Japanese researchers treated patients with “cycles” up to four times a week apart. Radio-sensitivity may be achieved with hyperthermia, and using heat with every radiation treatment may drive the treatment schedule.

One of the challenges in thermal therapy is delivering the appropriate amount of heat to the correct part of the patient's body. For this technique to be effective, the temperatures must be high enough, and the temperatures must be sustained long enough, to damage or kill the cancer cells. However, if the temperatures are too high, or if they are kept elevated for too long, then serious side effects, including death, can result. The smaller the place that is heated, and the shorter the treatment time, the lower the side effects. Conversely, tumor treated too slowly or at too low a temperature will not achieve therapeutic goals. The human body is a collection of tissues with differing heat capacities, all connected by a dynamic circulatory system with variable relationship to skin or lung surfaces designed to shed heat energy. All methods of inducing higher temperature in the body are countered by the thermo-regulatory mechanisms of the body. The body as a whole relies mostly on simple radiation of energy to the surrounding air from the skin (50% of heat lost this way) which is augmented by convection (blood shunting) and vaporization through sweat and respiration. Regional methods of heating may be more or less difficult based on the anatomic relationships, and tissue components of the particular body part being treated. Measuring temperatures in various parts of the body may be very difficult, and temperatures may locally vary even within a region of the body.

There is the further difficulty inherent in the devices delivering energy. Regional devices may not uniformly heat a target area, even without taking into account compensatory mechanisms of the body. A great deal of current research focuses on how one might precisely position heat-delivery devices (catheters, microwave and ultrasound applicators, etc.) using ultrasound or magnetic resonance imaging, as well as developing new types of nanoparticles that can more evenly distribute heat within a target tissue.

The thermo-acoustic (TA) effect refers to the generation of acoustic waves by electromagnetic (EM) irradiation, such as optical or microwave/radio frequency waves. In the past ten years, thermo-acoustic tomography (TAT) using pulsed EM excitation has undergone tremendous growth. Energy deposition inside biological tissue through the absorption of incident EM pulses will create a transient temperature rise. In the thermo-elastic mechanism of acoustic generation, a sound or stress wave is produced as a consequence of the expansion induced by the temperature variation. Thermo-acoustic signals are temperature dependent, which is an ideal characteristic for use in monitoring biological tissue temperature.

In the natural sciences, the term diathermy means “electrically induced heat” using high-frequency electromagnetic currents as a form of physical or occupational therapy. Diathermy is commonly used for muscle relaxation. It is also a method of heating tissue electromagnetically or ultrasonically for therapeutic purposes in medicine. Diathermy is used in physical therapy and occupational therapy to deliver moderate heat directly to pathologic lesions in the deeper tissues of the body.

Diathermy, whether achieved using short-wave radio frequency (range 1-100 MHz) or microwave energy (typically 915 MHz or 2.45 GHz), exerts physical effects and elicits a spectrum of physiological responses, the two methods differing mainly in their penetration capability. Different levels of penetration into living tissue can be achieved based upon selection of different frequencies of electromagnetic waves or based upon selection of a plurality of different frequencies of electromagnetic waves.

There are three forms of diathermy typically employed by physical and occupational therapists are ultrasound, short wave and microwave. The application of moderate heat by diathermy increases blood flow and speeds up metabolism and the rate of ion diffusion across cellular membranes.

Ultrasound diathermy employs high-frequency acoustic vibrations which, when propelled through the tissues, are converted into heat. This type of diathermy is especially useful in the delivery of heat to selected musculatures and structures because there is a difference in the sensitivity of various fibers to the acoustic vibrations; some are more absorptive and some are more reflective. For example, in subcutaneous fat, relatively little energy is converted into heat, but in muscle tissues there is a much higher rate of conversion to heat. The therapeutic ultrasound apparatus generates a high-frequency alternating current, which is then converted into acoustic vibrations. The apparatus is moved slowly across the surface of the part being treated. Ultrasound is a very effective agent for the application of heat.

Short wave diathermy machines use two condenser plates that are placed on either side of the body part to be treated. Another mode of application is by induction coils that are pliable and can be molded to fit the part of the body under treatment. As the high-frequency waves travel through the body tissues between the condensers or the coils, they are converted into heat. The degree of heat and depth of penetration depend in part on the absorptive and resistance properties of the tissues that the waves encounter. Short wave diathermy operations typically use the band frequencies of 13.56, 27.12, and 40.68 megahertz. Most commercial machines operate at a frequency of 27.12 MHz, a wavelength of approximately 11 meters.

Microwave diathermy uses microwaves, radio waves which are higher in frequency and shorter in wavelength than the short waves referenced above. Microwaves, which are also used in radar, have a frequency above 300 MHz and a wavelength less than one meter. Most, if not all, of the therapeutic effects of microwave therapy are related to the conversion of energy into heat and its distribution throughout the body tissues. This mode of diathermy is considered to be the easiest to use, but the microwaves typically have a relatively poor depth of penetration. Hyperthermia induced by microwave diathermy can raise the temperature of deep tissues from 41° C. to 45° C. using electromagnetic power.

Microwave diathermy is used in the management of tumors with conventional radiotherapy and chemotherapy. Hyperthermia has been used in oncology for more than 35 years, in addition to radiotherapy, in the management of different tumors. In 1994, hyperthermia has been introduced in several countries of the European Union as a modality for use in physical medicine and sports traumatology. Its use has been successfully extended to physical medicine and sports traumatology in Central and Southern Europe.

Diathermy is also a therapeutic treatment commonly prescribed for joint conditions such as rheumatoid arthritis and osteoarthritis. In diathermy, a high-frequency electric current is delivered via shortwave, microwave, or ultrasound to generate deep heat in body tissues.

Hyperthermia or thermotherapy or thermal therapy or diathermy are exemplary methods for elevating the temperature of a plurality of cells in a body, in specific areas in a body, or directed to specific levels of penetration into a body.

By itself, hyperthermia alone has demonstrated the ability to treat cancer. It is known that it significantly increases the effectiveness of other treatments.

When combined with radiation, hyperthermia is particularly effective at increasing the damage to acidic, poorly oxygenated parts of a tumor, and cells that are preparing to divide. Hyperthermia treatment is most effective when provided at the same time, or within an hour, of the radiation.

In the past decade hyperthermia treatments in conjunction with radiation have been used with curative intent in patients with early stage cancers of the breast, head and neck, and prostate. According to peer-reviewed scientific publications, Hyperthermia treatment has shown an improvement of 38% (53% vs. 15%) in Bladder Cancer when combined with Chemotherapy versus chemotherapy alone. In Breast cancer patients, an article by Vernon, et al in 1996 showed an improved response of 18% (59% vs. 41%) when radiation treatment was combined with Hyperthermia, and a 41% response in patients treated with radiation alone. Other cancer types that show a significant clinical response have been: Melanoma and Skin Cancer, Soft Tissue Sarcoma, Bladder, Cervical, Prostate, Rectal, Axilla and Chest Wall as well as recurrent or previously irradiated cancers.

Whole-body hyperthermia has yet to be practically combined with radiation, but it may be useful for chemotherapy and immunotherapy.

A body of research postulates that overheating the tumor cells is meant to create a lack of oxygen so that the heated tumor cells become over acidified, which leads to a lack of nutrients in the tumor. This in turn disrupts the metabolism of the cells so that cell death (apoptosis) can set in. In certain cases, chemotherapy or radiation that has previously not had any effect can be made effective. Because hyperthermia alters the cell walls by means of so-called heat shock proteins, cancer cells then react very much more effectively to the cytostatic type treatment and radiation. If hyperthermia is used conscientiously, it has no serious side effects. However whole body hyperthermia or local hyperthermia has better results when associated with infusion therapies (such as Vitamin C, B17, or Oxygen) and can be combined with an insulin potentiation therapy as a way to administrate a low dose chemotherapy.

Research in Russia has shown interesting results with extreme hyperthermia (body temperatures of 43.5 to 44° C. (110.3 to 111.2° F.)) where hyperthermia is used in cancer therapy, HIV therapy and Virus and Immune system illnesses. In a clinical trial, 30 patients received 4 extreme hyperthermia sessions within 70 days and 200 days later. All 30 patients showed fewer viruses) and an increase in CAD4+ (45%), which is 20 times higher when compared with the HAART Therapy.

The application of heat to treat certain conditions, including possible tumors, has a long history. Ancient Greeks, Romans, and Egyptians used heat to treat breast masses; this is still a recommended self-care treatment for breast engorgement. Medical practitioners in ancient India used regional and whole-body hyperthermia as treatments.

During the 19th century, tumor shrinkage after a high fever due to infection had been reported in a small number of cases. Typically, the reports documented the rare regression of a soft tissue sarcoma after erysipelas (an acute streptococcus bacterial infection of the skin; a different presentation of an infection by “flesh-eating bacteria”) was noted. Efforts to deliberately recreate this effect led to the development of Coley's toxin. A sustained high fever after induction of illness was considered critical to treatment success. This treatment is generally considered both less effective than modern treatments and, when it includes live bacteria, inappropriately dangerous.

Around the same period Westermark used localized hyperthermia to produce tumor regression in patients. Encouraging results were also reported by Warren when he treated patients with advanced cancer of various types with a combination of heat, induced with pyrogenic substance, and x-ray therapy. Out of 32 patients, 29 improved for 1 to 6 months.

External, localized hyperthermia treatments sensitize tumors to radiation and chemotherapy, making those treatments more effective. This device uses ultrasound technology to create heat at superficial and deep tumor sites. Hyperthermia may be combined with gene therapy, particularly using the heat shock protein 70 promoter.

Two major technological challenges make hyperthermia therapy complicated: the ability to achieve a uniform temperature in a tumor, and the ability to precisely monitor the temperatures of both the tumor and the surrounding tissue. Advances in devices to deliver uniform levels of the precise amount of heat desired, and devices to measure the total dose of heat received, are hoped for.

In locally advanced adenocarcinoma of middle and lower rectum, regional hyperthermia added to chemo-radiotherapy achieved good results in terms of rate of sphincter sparing surgery. External-beam radiation therapy is the most common type of radiation treatment, and it involves giving radiation from a machine located outside the body. It can treat large areas of the body, if necessary. The machine typically used to create the radiation beam is called a linear accelerator. Computers and software are used to adjust the size and shape of the beam and to direct it to target the tumor while attempting to avoid radiation of the healthy tissue that surrounds the cancer cells.

One embodiment may be a controlled heating apparatus for damaging or killing cancerous cells in a living human body, the apparatus being configured for: heating a portion of the living human body surrounding cancerous cells, such that the cancerous cells are damaged or killed, while detecting and monitoring a temperature of the cancerous cells; wherein glucose has been introduced to the portion of the living human body thereby raising the temperature of the cancerous cells more than a temperature of non-cancerous cells; wherein the heating is being carried out by bombardment of radiofrequency energy or ultrasonic energy to heat the cancerous cells containing the glucose to a temperature that is above a critical temperature that damages or kills the cancerous cells while keeping the non-cancerous cells below the critical temperature to avoid damaging or killing the non-cancerous cells. The apparatus may be configured for heating of the portion of the human body by directing radio frequency waves into the portion of the human body, wherein specific frequencies of the radio waves may be selected based upon a desired level of penetration. The glucose may be introduced by injection. The radio frequency waves may comprise radio waves containing a number of different frequencies having a power level at each of the frequencies which provide a depth of penetration of the radio waves into the living human body at each frequency resulting in an even heating of the portion of the human body at a corresponding number of different depths. The apparatus may further comprise an ultrasonic diathermy machine or a microwave diathermy machine.

Another embodiment may be a controlled heating apparatus for damaging or killing cancerous cells in a living human body, the apparatus being configured for: heating a portion of the living human body surrounding cancerous cells, such that the cancerous cells are damaged or killed, while detecting and monitoring a temperature of the cancerous cells; wherein the temperature of the cancerous cells is greater than a temperature of non-cancerous cells; wherein the heating is being carried out by bombardment of radiofrequency energy or ultrasonic energy to heat the cancerous cells to a temperature that is above a critical temperature that damages or kills the cancerous cells while keeping the non-cancerous cells below the critical temperature to avoid damaging or killing the non-cancerous cells. The apparatus may be configured for heating of the portion of the human body by directing radio frequency waves into the portion of the human body, wherein specific frequencies of the radio waves may be selected based upon a desired level of penetration. The radio frequency waves may comprise radio waves containing a number of different frequencies having a power level at each of the frequencies which provide a depth of penetration of the radio waves into the living human body at each frequency resulting in an even heating of the portion of the human body at a corresponding number of different depths. The apparatus may further comprise an ultrasonic diathermy machine or a microwave diathermy machine.

One embodiment may be a system for damaging or killing cancer cells in a living human body comprising: a temperature monitoring apparatus; and a controlled energy emitting apparatus; wherein the temperature monitoring apparatus is configured to monitor a plurality of temperatures in a human body, wherein the plurality of monitored temperatures comprises a temperature of a cancerous cells region and a temperature of a therapy region; wherein the therapy region comprises the cancerous cells region and a plurality of tissue of the human body that approximately surrounds the cancerous cells region; wherein the controlled energy emitting apparatus is configured to emit an energy that is directed to at least the cancerous cells region; and wherein the energy is configured to be changed in response to monitoring of the plurality of monitored temperatures. The plurality of monitored temperatures may further comprise the human body and a potential region of cancer. The potential region of cancer comprises a plurality of tissue of the human body that is approximately adjacent to the cancerous cells region. The temperature monitoring apparatus may be configured to control the controlled energy emitting apparatus to allow for one or more changes to the energy in response to one or more temperature changes of the plurality of monitored temperatures. The one or more changes to the energy that is controlled by the temperature monitoring apparatus may be made automatically based on an algorithm that takes into account the one or more temperature changes of the plurality of monitored temperatures. The one or more changes to the energy may comprise one or more of: increase, decrease, turn on, and turn off. The temperature monitoring apparatus may comprise one or more contact sensors. The temperature monitoring apparatus may comprise one or more contactless sensors. The temperature monitoring apparatus may comprise one or more advanced imaging technique devices. The energy may be a radiation treatment. The energy may be a hyperthermy treatment. The energy may be an electromagnetic wave hyperthermia therapy. An organic material may be applied to the cancerous cells region, which may be a glucose solution. In some embodiments the system may further comprise: at least one remote controller; wherein the at least one remote controller may be in communication with the temperature monitoring apparatus and the controlled energy emitting apparatus; and wherein the at least one remote controller may be configured to control the controlled energy emitting apparatus to change the energy in response to one or more temperature changes of the plurality of monitored temperatures. The controlled energy emitting apparatus may be configured to emit an energy that may be directed to the therapy region. The monitoring of the plurality of monitored temperatures may substantially prevent the temperature of the plurality of tissue of the human body that approximately surrounds the cancerous cells region from rising above 44 C while the temperature of the cancerous cells region is above 44 C. The controlled energy emitting apparatus heats the cancerous cells region to a temperature that is above a critical temperature that damages or kills the cancerous cells while keeping a plurality of non-cancerous cells in the plurality of tissue of the human body that approximately surrounds the cancerous cells region below the critical temperature to avoid damaging or killing the non-cancerous cells. It can do this because the tumor is hotter, naturally or raised by an injection of an organic substance, such as glucose, than the surrounding non-cancerous cells.

Another embodiment may be a temperature monitoring apparatus comprising: one or more advanced imaging technique devices; and a controller/communication module; wherein the one or more advanced imaging technique devices are configured to monitor a plurality of temperatures in a human body, wherein the plurality of monitored temperatures comprises a temperature of a cancerous cells region and a temperature of a therapy region; wherein the therapy region comprises the cancerous cells region and a plurality of tissue of the human body that approximately surrounds the cancerous cells region; wherein the controller/communication module is configured to control a controlled energy emitting apparatus that is configured to emit an energy that is directed at least the cancerous cells region; and wherein the controller/communication module is configured to make one or more changes to the energy in response to one or more changes in the plurality of monitored temperatures. The one or more changes to the energy that are made by the controller/communication module of the temperature monitoring apparatus may be configured to be done automatically based on the one or more changes in the plurality of monitored temperatures. The one or more changes to the energy may comprise one or more of: increase, decrease, turn on, and turn off. The energy may be a radiation treatment. The energy may be a hyperthermy treatment. The energy may be an electromagnetic wave hyperthermia therapy. An organic material may be applied to the cancerous cells. The organic material may be a glucose solution. The temperature monitoring apparatus may further comprise: at least one remote controller; wherein the at least one remote controller may be in communication with the temperature monitoring apparatus and the controlled energy emitting apparatus; and wherein the at least one remote controller may be also configured to control the controlled energy emitting apparatus to change the energy in response to the one or more changes to the plurality of monitored temperatures. The controlled energy emitting apparatus may be configured to emit the energy at the therapy region. Monitoring of the plurality of monitored temperatures may substantially prevent the temperature of the plurality of tissue of the human body that approximately surrounds the cancerous cells region from rising above 44 C while the temperature of the cancerous cells region is above 44 C. The monitoring of the plurality of monitored temperatures may substantially prevent the temperature of the plurality of tissue of the human body that approximately surrounds the cancerous cells region from rising above a critical temperature that damages or kills the plurality of tissue of the human body that approximately surrounds the cancerous cells region while the temperature of the cancerous cells region is above the critical temperature that damages or kills a plurality of cancerous cells of the cancerous cells region. It can do this because the tumor is hotter, naturally or raised by an injection of an organic substance, such as glucose, than the surrounding non-cancerous cells.

Another embodiment may be a system for damaging or killing cancer cells in a living human body comprising: a temperature monitoring apparatus; a controlled energy emitting apparatus; and at least one remote controller; wherein the temperature monitoring apparatus comprises a controller/communication module; wherein the temperature monitoring apparatus is configured to monitor a plurality of temperatures in a human body, wherein the plurality of monitored temperatures comprises a temperature of a cancerous cells region and a temperature of a therapy region; wherein the therapy region comprises the cancerous cells region and a plurality of tissue of the human body that approximately surrounds the cancerous cells region; wherein the controlled energy emitting apparatus is configured to emit an energy that is directed to at least the cancerous cells region; wherein the controller/communication module and the remote controller are configured to control the controlled energy emitting apparatus; and wherein the controller/communication module and the remote controller may be configured to make one or more changes to the energy in response to one or more changes in the plurality of monitored temperatures. The plurality of monitored temperatures may further comprise the human body and a potential region of cancer; wherein the potential region of cancer may comprise a plurality of tissue of the human body that is approximately adjacent to the cancerous cells region. The one or more changes to the energy may be made automatically based on the one or more temperature changes of the plurality of monitored temperatures. The one or more changes to the energy may comprise one or more of: increase, decrease, turn on, and turn off. The temperature monitoring apparatus may further comprise one or more contact sensors, one or more contactless sensors, or one or more advanced imaging technique devices. The energy may be a radiation treatment. The energy may be a hyperthermy treatment. The energy may be an electromagnetic wave hyperthermia therapy. An organic material may be applied to the cancerous cells region. The organic material may be a glucose solution. The controlled energy emitting apparatus may be configured to emit the energy at the therapy region. The monitoring of the plurality of monitored temperatures may substantially prevent the temperature of the plurality of tissue of the human body that approximately surrounds the cancerous cells region from rising above 44 C while the temperature of the cancerous cells region is above 44 C. The monitoring of the plurality of monitored temperatures may substantially prevent the temperature of the plurality of tissue of the human body that approximately surrounds the cancerous cells region from rising above a critical temperature that damages or kills the plurality of tissue of the human body that approximately surrounds the cancerous cells region while the temperature of the cancerous cells region is above the critical temperature that damages or kills a plurality of cancerous cells of the cancerous cells region. It can do this because the tumor is hotter, naturally or raised by an injection of an organic substance, such as glucose, than the surrounding non-cancerous cells.

The contents of this summary section are provided only as a simplified introduction to the disclosure and are not intended to be used to limit the scope of the claims. These, as well as other components, steps, features, objects, benefits, and advantages, will now become clear from a review of the following detailed description of illustrative embodiments, and of the claim.

### BRIEF DESCRIPTION OF THE SEVERAL VIEWS OF THE DRAWINGS

The drawings show illustrative embodiments, but do not depict all embodiments. Other embodiments may be used in addition to or instead of the illustrative embodiments. Details that may be apparent or unnecessary may be omitted for the purpose of saving space or for more effective illustrations. Some embodiments may be practiced with additional components or steps and/or without some or all components or steps provided in the illustrations. When different drawings contain the same numeral, that numeral refers to the same or similar components or steps.

FIG. 1 is an illustrative diagram of one embodiment of the system for damaging cancerous cells by application of energy while monitoring the temperatures of cancerous cells and the surrounding tissue.

FIG. 2 is an illustrative diagram of another embodiment of the system for damaging cancerous cells by application of energy while monitoring the temperatures of cancerous cells and the surrounding tissue.

FIG. 3 is an illustrative diagram of embodiment of the system for damaging cancerous cells by application of energy while monitoring the temperatures of cancerous cells and the surrounding tissue.

### DETAILED DESCRIPTION OF THE SEVERAL VIEWS OF THE DRAWING

Before the present device, methods, and systems are disclosed and described, it is to be understood that the methods and systems are not limited to specific device and methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in disclosed methods. Thus, if there are a variety of additional steps that may be performed it is understood that each of these additional steps may be performed with any specific embodiment or combination of embodiments of the disclosed methods.

The present methods and systems may be understood more readily by reference to the following detailed description of preferred embodiments and the examples included therein and to the Figures and their previous and following description.

In the following description, certain terminology is used to describe certain features of one or more embodiments. For purposes of the specification, unless otherwise specified, the term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item, or result.

As used herein, the terms “approximately” and “about” generally refer to a deviance of within 5% of the indicated number or range of numbers. In one embodiment, the term “approximately” and “about”, may refer to a deviance of between 0.001-40% from the indicated number or range of numbers.

As used herein, “ul” refers to microliter, “ml” refers to milliliter, and “ng” refers to nanogram.

Various embodiments are now described with reference to the drawings. In the following description, for purposes of explanation, numerous specific details are set forth in order to provide a thorough understanding of one or more embodiments. It may be evident, however, that the various embodiments may be practiced without these specific details. In other instances, well-known structures and devices are shown in block diagram form to facilitate describing these embodiments.

Various embodiments presented in terms of systems may comprise a number of components, modules, and the like. It is to be understood and appreciated that the various systems may include additional components, modules, etc. and/or may not include all of the components, modules, etc. discussed in connection with the figures. A combination of these approaches may also be used.

The temperature monitoring, which includes taking temperature measurements, may be accomplished by probes, sensors, or advanced imaging techniques. The temperature monitoring of the tumor and surrounding tissue may preferably be done in real time during a heat treatment via the probes, sensors, and/or advanced imaging techniques.

Generally, temperature sensors measure the amount of heat or energy that is generated by an object or system, allowing observers to detect any change to that temperature by producing either an analogue or digital output. There are many different types of temperature sensors that may be used. Various temperature sensors may have different characteristics depending upon their structure and application. The two basic temperature sensor types are contact and contactless. A contact temperature sensor requires physical contact with the object being sensed and they generally may use conduction to monitor changes in temperature. Non-contact temperature sensors may use convection and/or radiation to monitor changes in temperature. Contact temperature sensors may include temperature probes.

Generally, there are three groups of temperature sensors, electro-mechanical (such as a thermostat, bimetallic thermostat), resistive (thermistor, resistive temperature detector), and electronic (thermocouple). Other types and groups of sensors may comprise semiconductor junction sensors, infra-red and thermal radiation sensors, medical type thermometers, indicators and color changing inks or dyes. The temperature monitor of the present disclosure may use any type or group of temperature sensors.

Because tumors may be at a temperature that is elevated with respect to the surrounding non-cancerous tissue, if the tumor and the surrounding tissue are heated up, the tumor which is already hotter, will rise to a higher temperature than the surrounding non-cancerous tissue. Eventually, the tumor will be heated high enough to kill the tumor/cancerous cells, but the surrounding non-cancerous tissue will not be heated high enough to be killed.

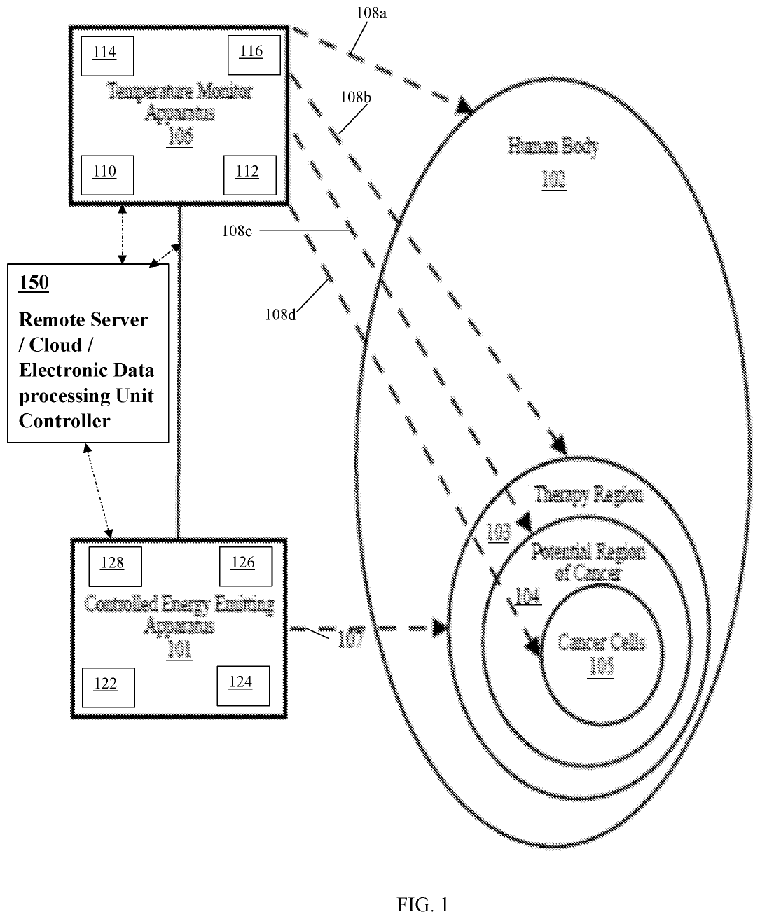

FIG. 1 is an illustrative diagram of one embodiment of the system for damaging cancerous cells by application of energy while monitoring the temperatures of cancerous cells and the surrounding tissue. As shown in FIG. 1, the system may comprise a temperature monitor apparatus 106, remote electronic data processing unit controller 150 (remote controller), and a controlled energy emitting apparatus 101. The controlled energy emitting apparatus may be controlled by temperature monitor apparatus 106 or the remote controller 150. The controlled energy emitting apparatus may apply energy to the entirety of the cancer cells 105 so that the cancer cells 105 are damaged or killed, but the surrounding tissue in the human body 102, 103 is not damaged or killed. As shown in FIG. 1, a human body 102 may have or is suspected of having cancer cells within an area 105. The cancerous area 105 may be within a region 104 identified as a potential region of cancer. A therapy region 103, which may surround regions 104, 105, may have energy 107 applied to it.

In one embodiment, the energy 107 can be a form of radiation, hyperthermy, or electromagnetic wave treatment, such as radionuclide therapy, proton therapy, laser therapy, light, heat, x-ray therapy, magnetic resonance thermography, infrared thermography, radionuclide therapy, proton therapy, hyperthermy, and/or electromagnetic wave therapy.

FIG. 1 shows that the temperature monitor apparatus 106 may comprise contact temperature sensors (such as probes) 110, contactless temperature sensors 112, advanced imaging technique devices 114, and controller/communication module 116. The temperature monitor apparatus 106 may be configured to monitor the temperatures of one, more, or, as preferred all of, the body 102, the therapy region 103, the potential region of cancerous cells 104, and cancerous cells 105. The monitoring of the temperature 108a of the body 102 may be done via contact temperature sensors 110, contactless temperature sensors 112, and or advanced imaging technique devices 114. The monitoring of the temperature 108b of the therapy region 103 may be done via contact temperature sensors 110, contactless temperature sensors 112, and or advanced imaging technique devices 114. The monitoring of the temperature 108c of the potential region of cancer 104 may be done via contact temperature sensors 110, contactless temperature sensors 112, and or advanced imaging technique devices 114. The monitoring of the temperature 108d of the cancerous cells 105 may be done via contact temperature sensors 110, contactless temperature sensors 112, and or advanced imaging technique devices 114.

The controller/communication module 116 may be configured to allow the temperature monitor apparatus 106 to dynamical monitor/measure the temperatures 108a-d, record the temperatures, and display the temperatures to a user. The displaying of the temperatures 108a-d may be one via a display that is part of the controller/communication module 116, or by communicating the temperatures 108a-d to a remote device, such as remote controller 150, which itself may be a smart phone. In addition to monitoring temperatures 108a-d, the controller/communication module 116 may also communicate with an electronic data processing unit, and/or the controlled energy emitting apparatus 101. In various embodiments, either or both of the electronic data processing unit and the temperature monitor apparatus 106 may use the monitored temperature data to control the delivery of energy 107 from the controlled energy emitting apparatus 101 to the regions 103, 104, 105. The energy 107 may be turned off, turned on, turned up, turned down, or the type of energy delivered by be changed (radiation v. radiant heat, for example). This allows the system to keep the temperature of the tissue surrounding the cancerous cells from experiencing cellular death, even though the tumor is killed via the monitoring of the temperatures 108a-d, the temperatures of regions 102 and 103 may be kept under 44° C. (111° F.).

The monitoring allows this to happen even though the temperature of regions 105 and 104 may exceed 44° C. (111° F.), which causes death to the tumor cells. In some embodiments it may be that the tumor might have a critical temperature that damages or kills them that is below 44 C. If that is the case, then the temperature of the tumor may be raised until that critical temperature is reached. The monitoring ensures that the surrounding tissue is kept at a lower temperature than the tumor and does not go above the critical temperature that damages or kills those non-cancerous cells of the surrounding tissue. In some cases, the tumor might have a critical temperature that damages or kills them that is above 45 C. If that is the case, then the temperature of the tumor may be raised until that critical temperature is reached. The monitoring ensures that the surrounding tissue is kept at a lower temperature than the tumor and does not go above the critical temperature that damages or kills those non-cancerous cells of the surrounding tissue, which may be 44C, higher, or lower.

Remote controller 150 may be a dedicated piece of hardware or may be a computer, such as a laptop or smartphone, that is running specific software that allows it to communicate with the temperature monitor apparatus 106 and/or controlled energy emitting apparatus 101. The remote controller 150 may also be a server or otherwise cloud based. The remote controller 150 may have a display and user interface (touch screen, mouse, keyboard, and the like) that allows a user to monitor the temperatures 108a-d and then control or modify the energy 107 in order to kill the cells in regions 104, 105, but not kill the cells in regions 102, 103. In some embodiments the temperature monitor apparatus 106 and/or the remote controller 150 may have a program that automatically adjusts the energy 107 in response to the monitored temperatures 108a-d.

As shown in FIG. 1, the controlled energy emitting apparatus 101 may have communication module 122, which allows it to communicate with, and/or be controlled by, apparatus 106 and remote controller 150. The controlled energy emitting apparatus 101 may have a hyperthermy emitter 124 and/or radiation emitter 126 for providing energy 107 to a therapy region 103. The controlled energy emitting apparatus 101 may have power source 128, which may be substantially any source of power, including a battery.

The devices of the system may be in wired or wireless communication.

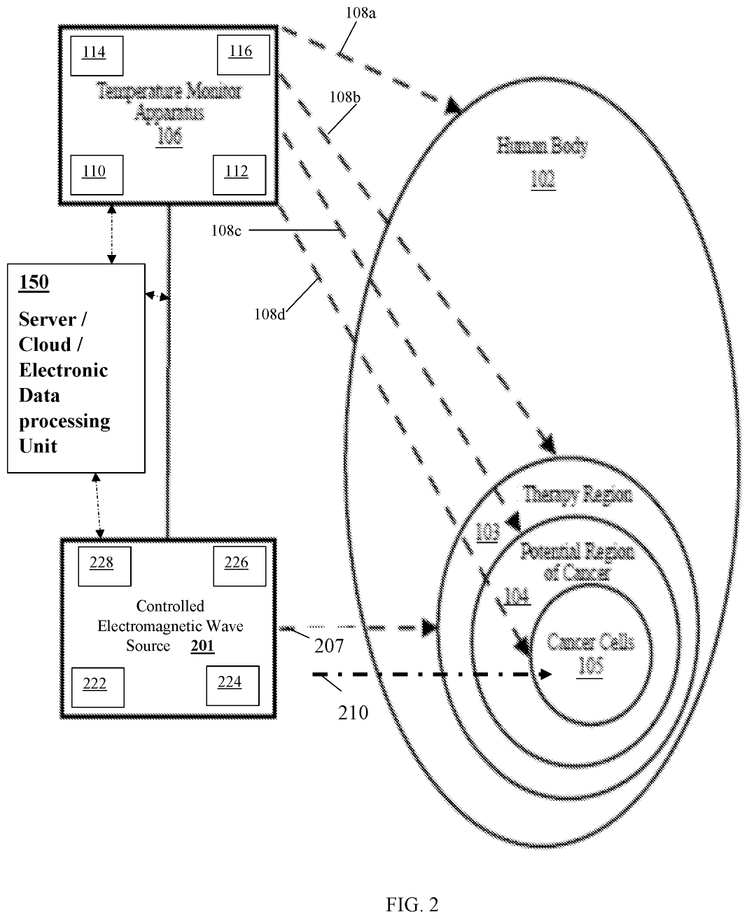

FIG. 2 is an illustrative diagram of another embodiment of the system for damaging cancerous cells by application of energy while monitoring the temperatures of cancerous cells and the surrounding tissue. FIG. 2 is an illustrative diagram of another embodiment of the present disclosure in which the temperature monitor apparatus 106 and the remote controller 150 are the same as shown and described in FIG. 1, but they are in communication with a controlled electromagnetic wave source 201, which may be configured to bombard the cancerous cells 105 (and/or regions 103 and 104) with energy via electromagnetic waves 207 so that the cancer cells 105 are damaged or killed, but the surrounding living tissue 102, 103 is not damaged or killed. This heating of the temperature 108c via electromagnetic waves 207 of the potential region 104 of cancerous cells above a normal body temperature raises the temperature of the cancerous cells 105 to a temperature 108d at which the tumor 105 are damaged or killed. The temperature monitor apparatus 106 operates to monitor and or measure variations in the temperatures 108a-d of the human body 102, the heated region 103, the potential region of cancer 104, and the cancer cells 105. The temperature monitor apparatus 106 then provides variational feedback to the controlled electromagnetic wave source 201, allowing the controlled electromagnetic wave source 201 to target the cancerous cells 105 more efficiently. The depth of penetration 210 of the electromagnetic waves 207 may be adjusted by selecting a frequency, via the frequency settings 224 for the electromagnetic waves 207 produced by electromagnetic source 201.

In another embodiment, the controlled electromagnetic wave source 201 emits electromagnetic waves 207 from emitter 226 at a plurality of selected frequencies 224, thus causing the cancerous cells 105 to be heated by waves 207 to a temperature (typically over 111 C) that causes destruction or damage to at least a portion of the cancerous cells 105. The frequencies of the plurality of frequencies 224 are optionally selected based upon prior predetermined measurements and studies carried out in a conventional manner to determine a predicted level or depth of penetration 210 into living tissue, and then selecting one or more of a plurality of frequencies to achieve maximum heating in the area or at the depth of penetration 210 known to contain the cancerous cells 105.

The controlled electromagnetic wave source 201 may have a communications module 222 and power source 228, which may be substantially similar to the communications module 122 and power source 128.

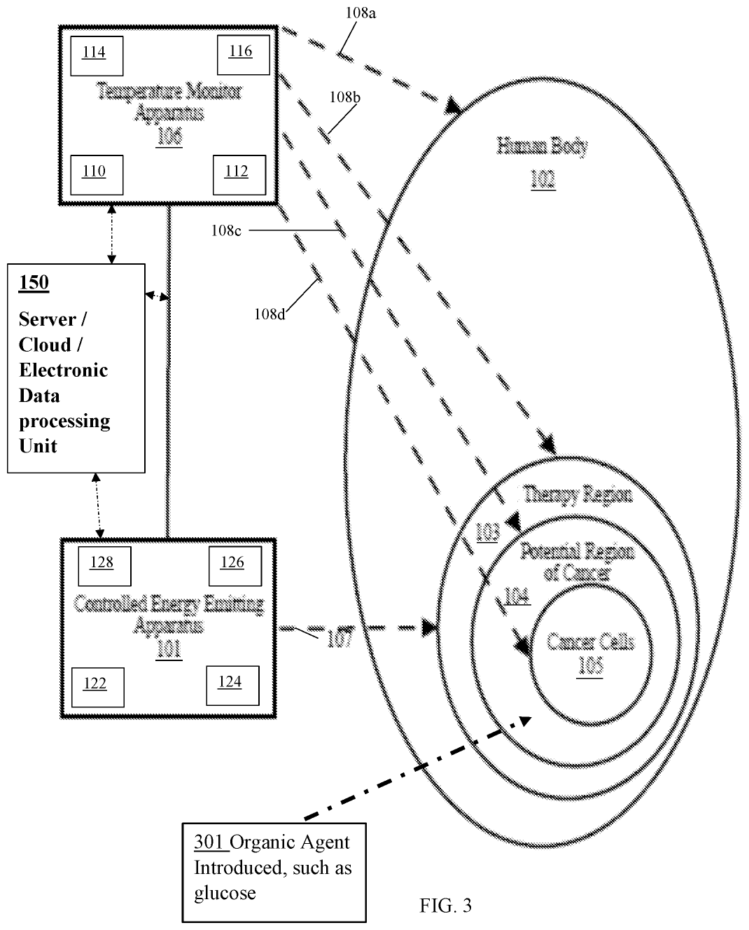

FIG. 3 is an illustrative diagram of embodiment of the system for damaging cancerous cells by application of energy while monitoring the temperatures of cancerous cells and the surrounding tissue. FIG. 3 is essentially identical to FIG. 1, but shows the addition of the optional element of an infusion (or injection) of an organic agent 301, such as glucose, or any other organic agent that induces exothermic heating of cancerous cells. The organic agent 301 application may act as a temperature biasing component, causing an increase in the temperature of the cancerous cells 105 as a result of hyperactive metabolism of certain organic agents in cancerous tissues. This in turn allows the temperature monitor apparatus 106 to more precisely locate the cancer cells 105 for the controlled energy emitting apparatus 101. Organic agents can also be used with electromagnetic wave. Because the temperature of the cancer cells 105 has increased relative to the surrounding human body tissue 102, the cancer cells 105 are more easily perceptible to the temperature monitor apparatus 106 and are more susceptible to the applied energy 107 from the controlled energy emitting apparatus 106.

The foregoing description of the preferred embodiment has been presented for the purposes of illustration and description. While multiple embodiments are disclosed, still other embodiments will become apparent to those skilled in the art from the above detailed description. These embodiments are capable of modifications in various obvious aspects, all without departing from the spirit and scope of protection. Accordingly, the detailed description is to be regarded as illustrative in nature and not restrictive. Also, although not explicitly recited, one or more embodiments may be practiced in combination or conjunction with one another. Furthermore, the reference or non-reference to a particular embodiment shall not be interpreted to limit the scope of protection. It is intended that the scope of protection not be limited by this detailed description, but by the claims and the equivalents to the claims that are appended hereto.

Except as stated immediately above, nothing that has been stated or illustrated is intended or should be interpreted to cause a dedication of any component, step, feature, object, benefit, advantage, or equivalent, to the public, regardless of whether it is or is not recited in the claims.

The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those of ordinary skill in the art that various modifications and variations may be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A controlled heating apparatus for damaging or killing cancerous cells in a living human body, said apparatus being configured for:
heating a portion of said living human body surrounding cancerous cells, such that said cancerous cells are damaged or killed, while detecting and monitoring a temperature of said cancerous cells;
wherein glucose has been introduced to said portion of said living human body thereby raising said temperature of said cancerous cells more than a temperature of non-cancerous cells;
wherein said heating is being carried out by bombardment of radiofrequency energy or ultrasonic energy to heat said cancerous cells containing said glucose to a temperature that is above a critical temperature that damages or kills said cancerous cells while keeping said non-cancerous cells below said critical temperature to avoid damaging or killing said non-cancerous cells.

2. The apparatus of claim 1, wherein said apparatus is configured for heating of said portion of said human body by directing radio frequency waves into said portion of said human body, wherein specific frequencies of said radio waves are selected based upon a desired level of penetration.

3. The apparatus of claim 1, wherein said glucose is introduced by injection.

4. The apparatus of claim 2, wherein said radio frequency waves comprise radio waves containing a number of different frequencies having a power level at each of the frequencies which provide a depth of penetration of said radio waves into said living human body at each frequency resulting in an even heating of said portion of said human body at a corresponding number of different depths.

5. The apparatus of claim 1, further comprising an ultrasonic diathermy machine.

6. The apparatus of claim 1, further comprising a microwave diathermy machine.

7. A controlled heating apparatus for damaging or killing cancerous cells in a living human body, said apparatus being configured for:
heating a portion of said living human body surrounding cancerous cells, such that said cancerous cells are damaged or killed, while detecting and monitoring a temperature of said cancerous cells;
wherein said temperature of said cancerous cells is greater than a temperature of non-cancerous cells;
wherein said heating is being carried out by bombardment of radiofrequency energy or ultrasonic energy to heat said cancerous cells to a temperature that is above a critical temperature that damages or kills said cancerous cells while keeping said non-cancerous cells below said critical temperature to avoid damaging or killing said non-cancerous cells.

8. The apparatus of claim 7, wherein said apparatus is configured for heating of said portion of said human body by directing radio frequency waves into said portion of said human body, wherein specific frequencies of said radio waves are selected based upon a desired level of penetration.

9. The apparatus of claim 8, wherein said radio frequency waves comprise radio waves containing a number of different frequencies having a power level at each of the frequencies which provide a depth of penetration of said radio waves into said living human body at each frequency resulting in an even heating of said portion of said human body at a corresponding number of different depths.

10. The apparatus of claim 7, further comprising an ultrasonic diathermy machine.

11. The apparatus of claim 7, further comprising a microwave diathermy machine.

12. A system for damaging or killing cancer cells in a living human body comprising:
a temperature monitoring apparatus; and
a controlled energy emitting apparatus;
wherein said temperature monitoring apparatus is configured to monitor a plurality of temperatures in a human body, wherein said plurality of monitored temperatures comprises a temperature of a cancerous cells region and a temperature of a therapy region;
wherein said therapy region comprises said cancerous cells region and a plurality of tissue of said human body that approximately surrounds said cancerous cells region;
wherein said controlled energy emitting apparatus is configured to emit an energy that is directed to at least said cancerous cells region; and
wherein said energy is configured to be changed in response to monitoring of said plurality of monitored temperatures.

13. The system of claim 12, wherein said plurality of monitored temperatures further comprise said human body and a potential region of cancer;
wherein said potential region of cancer comprises a plurality of tissue of said human body that is approximately adjacent to said cancerous cells region.

14. The system of claim 12, wherein said temperature monitoring apparatus is configured to control said controlled energy emitting apparatus to allow for one or more changes to said energy in response to one or more temperature changes of said plurality of monitored temperatures.

15. The system of claim 14, wherein said one or more changes to said energy controlled by said temperature monitoring apparatus are made automatically based on said one or more temperature changes of said plurality of monitored temperatures.

16. The system of claim 12, wherein said one or more changes to said energy comprise one or more of: increase, decrease, turn on, and turn off

17. The system of claim 12, wherein said temperature monitoring apparatus comprises one or more contact sensors.

18. The system of claim 12, wherein said temperature monitoring apparatus comprises one or more contactless sensors.

19. The system of claim 12, wherein said temperature monitoring apparatus comprises one or more advanced imaging technique devices.

20. The system of claim 12, wherein said energy is a radiation treatment.

21. The system of claim 12, wherein said energy is a hyperthermy treatment.

22. The system of claim 12, wherein said energy is an electromagnetic wave hyperthermia therapy.

23. The system of claim 12, wherein an organic material is applied to said cancerous cells region.

24. The system of claim 23, wherein said organic material is a glucose solution.

25. The system of claim 12, further comprising:
at least one remote controller;
wherein said at least one remote controller is in communication with said temperature monitoring apparatus and said controlled energy emitting apparatus; and
wherein said at least one remote controller is configured to control said controlled energy emitting apparatus to change said energy in response to one or more temperature changes of said plurality of monitored temperatures.

26. The system of claim 23, wherein said controlled energy emitting apparatus is configured to emit an energy that is directed to said therapy region.

27. The system of claim 12, wherein monitoring of said plurality of monitored temperatures substantially prevents said temperature of said plurality of tissue of said human body that approximately surrounds said cancerous cells region from rising above 44 C while said temperature of said cancerous cells region is above 44 C.

28. The system of claim 12, wherein said controlled energy emitting apparatus heats said cancerous cells region to a temperature that is above a critical temperature that damages or kills said cancerous cells while keeping a plurality of non-cancerous cells in said plurality of tissue of said human body that approximately surrounds said cancerous cells region below said critical temperature to avoid damaging or killing said non-cancerous cells.

29. A temperature monitoring apparatus comprising:
one or more advanced imaging technique devices; and
a controller/communication module;
wherein said one or more advanced imaging technique devices are configured to monitor a plurality of temperatures in a human body, wherein said plurality of monitored temperatures comprises a temperature of a cancerous cells region and a temperature of a therapy region;
wherein said therapy region comprises said cancerous cells region and a plurality of tissue of said human body that approximately surrounds said cancerous cells region;
wherein said controller/communication module is configured to control a controlled energy emitting apparatus that is configured to emit an energy that is directed at least said cancerous cells region; and
wherein said controller/communication module is configured to make one or more changes to said energy in response to one or more changes in said plurality of monitored temperatures.

30. The temperature monitoring apparatus of claim 29, wherein said one or more changes to said energy that are made by said controller/communication module of said temperature monitoring apparatus are configured to be done automatically based on said one or more changes in said plurality of monitored temperatures.

31. The temperature monitoring apparatus of claim 29, wherein said one or more changes to said energy comprise one or more of: increase, decrease, turn on, and turn off

32. The temperature monitoring apparatus of claim 29, wherein said energy is a radiation treatment.

33. The temperature monitoring apparatus of claim 29, wherein said energy is a hyperthermy treatment.

34. The temperature monitoring apparatus of claim 29, wherein said energy is an electromagnetic wave hyperthermia therapy.

35. The temperature monitoring apparatus of claim 29, wherein an organic material is applied to said cancerous cells.

36. The temperature monitoring apparatus of claim 35, wherein said organic material is a glucose solution.

37. The temperature monitoring apparatus of claim 36, further comprising:
at least one remote controller;
wherein said at least one remote controller is in communication with said temperature monitoring apparatus and said controlled energy emitting apparatus; and
wherein said at least one remote controller is also configured to control said controlled energy emitting apparatus to change said energy in response to said one or more changes to said plurality of monitored temperatures.

38. The temperature monitoring apparatus of claim 29, wherein said controlled energy emitting apparatus is configured to emit said energy at said therapy region.

39. The temperature monitoring apparatus of claim 38, wherein monitoring of said plurality of monitored temperatures substantially prevents said temperature of said plurality of tissue of said human body that approximately surrounds said cancerous cells region from rising above 44 C while said temperature of said cancerous cells region is above 44 C.

40. The temperature monitoring apparatus of claim 38, wherein monitoring of said plurality of monitored temperatures substantially prevents said temperature of said plurality of tissue of said human body that approximately surrounds said cancerous cells region from rising above a critical temperature that damages or kills said plurality of tissue of said human body that approximately surrounds said cancerous cells region while said temperature of said cancerous cells region is above said critical temperature that damages or kills a plurality of cancerous cells of said cancerous cells region.

41. A system for damaging or killing cancer cells in a living human body comprising:
a temperature monitoring apparatus;
a controlled energy emitting apparatus; and
at least one remote controller;
wherein said temperature monitoring apparatus comprises a controller/communication module;
wherein said temperature monitoring apparatus is configured to monitor a plurality of temperatures in a human body, wherein said plurality of monitored temperatures comprises a temperature of a cancerous cells region and a temperature of a therapy region;
wherein said therapy region comprises said cancerous cells region and a plurality of tissue of said human body that approximately surrounds said cancerous cells region;
wherein said controlled energy emitting apparatus is configured to emit an energy that is directed to at least said cancerous cells region;
wherein said controller/communication module and said remote controller are configured to control said controlled energy emitting apparatus; and
wherein said controller/communication module and said remote controller are configured to make one or more changes to said energy in response to one or more changes in said plurality of monitored temperatures.

42. The system of claim 41, wherein said plurality of monitored temperatures further comprise said human body and a potential region of cancer;
wherein said potential region of cancer comprises a plurality of tissue of said human body that is approximately adjacent to said cancerous cells region.

43. The system of claim 41, wherein said one or more changes to said energy are made automatically based on said one or more temperature changes of said plurality of monitored temperatures.

44. The system of claim 414, wherein said one or more changes to said energy comprise one or more of: increase, decrease, turn on, and turn off

45. The system of claim 41, wherein said temperature monitoring apparatus further comprises one or more contact sensors.

46. The system of claim 41, wherein said temperature monitoring apparatus further comprises one or more contactless sensors.

47. The system of claim 41, wherein said temperature monitoring apparatus further comprises one or more advanced imaging technique devices.

48. The system of claim 41, wherein said energy is a radiation treatment.

49. The system of claim 41, wherein said energy is a hyperthermy treatment.

50. The system of claim 41, wherein said energy is an electromagnetic wave hyperthermia therapy.

51. The system of claim 41, wherein an organic material is applied to said cancerous cells region.

52. The system of claim 41, wherein said organic material is a glucose solution.

53. The system of claim 41, wherein said controlled energy emitting apparatus is configured to emit said energy at said therapy region.

54. The system of claim 41, wherein monitoring of said plurality of monitored temperatures substantially prevents said temperature of said plurality of tissue of said human body that approximately surrounds said cancerous cells region from rising above 44 C while said temperature of said cancerous cells region is above 44 C.

55. The temperature monitoring apparatus of claim 41, wherein monitoring of said plurality of monitored temperatures substantially prevents said temperature of said plurality of tissue of said human body that approximately surrounds said cancerous cells region from rising above a critical temperature that damages or kills said plurality of tissue of said human body that approximately surrounds said cancerous cells region while said temperature of said cancerous cells region is above said critical temperature that damages or kills a plurality of cancerous cells of said cancerous cells region.
